(19) United States
(12) Patent Application Publication
(10) Pub. No.: US 2016/0242454 A1
(43) Pub. Date: Aug. 25, 2016

(54) AUTOMATICALLY ROTATING, HlGH-PRESSURE STERILIZER AND PROCESSING METHOD HAVING CONTINUOUS OPERATION STAGES

(71) Applicant: NAPASOL ASIA LTD., Wanchai, Hong Kong (CN)

(72) Inventor: Ursula WAENY, Zermatt (CH)

(73) Assignee: NAPASOL ASIA LTD, Wanchai, Hong Kong (CN)

(21) Appl. No.: 15/029,595

(22) PCT Filed: Oct. 13, 2014
(86) PCT No.: PCT/IB2014/002083

§ 371 Date: Apr. 14, 2016

(30) Foreign Application Priority Data
Oct. 15, 2013 (CN) 201310483188.5

(51) Int. Cl. A23L 3/16; A61L 2/07; A61L 2/00; A23B 7/005; A23B 9/02

(52) U.S. Cl. CPC A23L 3/165; A23B 7/0053; A23B 9/025; A61L 2/0023; A61L 2/07; A23V 2002/00

(57) ABSTRACT

Provided is an automatically rotating, high-pressure sterilizer 10. A vessel 110 thereof has a vertical axis 151 that inclines to a horizontal rotation axis 150. A processing chamber 114 in the vessel 110 has a smooth and non-protruding inner surface 114a or has a mechanical stirring element for stirring products 100. The vessel 110 comprises at least: components for supporting and rotating the vessel 140-144, a first connector 120, temperature-controlling components 121-123, processing chamber 114 supplying components 131-134, sensor components 145, 150, and a PLC-type electronic controller 163. Also provided is a corresponding processing method. The present invention achieves high quality while having low operation costs, and is suited for high production volumes.

Object of the present invention is an automatic rotating autoclave for the combined pasteurization and blending of organic materials in powder form or as particulates under saturated steam conditions; a further object of the invention is the treatment procedure of the said materials by the means of the said rotating autoclave.

### FIELD OF APPLICATION

The innovation is especially, but not exclusively, suitable for its application in the area of the industrial production of food products, finding also applications for the treatment of non-food products, for example in the sector of the cosmetic and pharmaceutical industry; the invention is particularly efficient for the treatment of bulk products in powder form or as particulates with a low moisture content, such as for example, but not limited to cereals, dried fruit, coffee or spices. Moreover, the invention is capable of combined treatments including blending, pasteurization or sterilization of any organic bulk material in powder form or as particulates, with a low moisture content.

Between the known and conventional treatments for the conservation of food products there are physical methods using heat at a specific temperature level and during a defined period of time, in a way to eliminate or inhibit the growth of microorganisms which generally are contaminating organic materials, such as for example, in the case of food products, bacteria, mold, yeast, and viruses. In particular, it is known that a specific temperature and the presence of moisture are among the factors which to a large extent are at the origin of the development of microorganisms; in the specific case of low moisture food products, such as for example dried fruit or cereals, the treatments which are mostly used for the reduction of the bacterial load depending on their thermal resistance, use the heating of the product to a high temperature level.

Between the known solutions which are generally used to reduce such bacterial load we recall specially the pasteurization and the sterilization. The process called pasteurization generally provides temperatures in the range between 60° C. and 100° C. during a time between 15 seconds and 30 minutes, depending on the defined cycle and on the applied temperature, the time being shorter at higher temperature levels; the different pasteurization methods substantially destroy pathogenic bacteria by denaturing the enzymes. The process conventionally called sterilization, on the other hand, is used to destroy more resistant microorganisms and spores, providing temperatures in the range between 100° C. and 150° C. during a time between a few seconds and over 20 minutes, the time being shorter at higher temperature levels.

In principle, the said pasteurization and sterilization treatments are technologically similar, using a system with a closed treatment chamber which allows the use of heat in a homogeneous and controlled way. In both cases the treatment chamber has to be hermetically closed and it has to be designed to work at a pressure range different from atmospheric pressure; such a machine configuration for pasteurization or sterilization is conventionally called autoclave.

Conventionally, the term pasteurization is used for any type of thermal treatment of organic products which aims for the extension of the shelf life of the product, destroying or inhibiting the development of said microorganisms as well as insects or other types of infestations. In the present description the generic term of pasteurization will be used in its widest signification, thus including the thermal sterilization treatment as conventionally understood.

Between the processes generally used in the food industry it is also remembered the blending, the particular objective of which is the mixing of different organic bulk products in powder form or as particulates and with a low moisture content, being of a nature different from each other or being of the same nature but of a different shape or particle size; as an example, such a process is generally used to achieve homogeneous mixtures of cereals, dried fruit or spice blends, and it is also used in the production of feed, and in the pharmaceutical and cosmetic industries. Most blending systems rely on the mechanical action of a paddle, screw, or scraper to mix the ingredients, other blenders of dry foods that do not use this mechanical action are based on the physical properties of gravity and rotation to obtain a homogenous mix of ingredients.

Furthermore, it is also known that the said pasteurization and the said blending are often performed both on the same products before packaging, not simultaneously, but successively in different machines; such machines, in fact, are based on different technologies: a pasteurizer provides a thermal treatment in a static, hermetically closed chamber, resulting in the reduction of the microbial load of the product; a blender, instead, is substantially a machine which mixes different products, generally through the rotation of the whole vessel which contains the products to be blended, or through rotating devices inside the vessel, such as for example paddles or spirals, sometimes mounted in series.

It is also known that conventional solutions, although widespread and standardized, present significant and unresolved production problems. In the case of the pasteurization or sterilization, such problems mainly concern the homogeneity of the treatment throughout a load and the optimized control of the entire cycle and of the respective process parameters, as well as the agglomeration of the product; in the case of the blending, on the other hand, the problems are mainly related to the mechanical damage resulting from agitating the product during the blending cycle. In addition to the before mentioned problems, it is also widely known that both of the said processing technologies present the serious risk of contamination during the loading and unloading phase, in addition to the general problem of cleaning of all the surfaces which are in contact with the product, particularly in the case of closed machines and when changing the products to be processed.

In the food industry the pasteurization or sterilization process is often performed at the end of the complete production process and immediately before the packaging of the product, with the objective to reduce as much as possible the risks related to food safety and to extend the shelf life of the product. This treatment is otherwise called a kill step.

Such a treatment can be performed advantageously by using steam; in order to achieve an optimized efficiency and to minimize the impact on the product characteristics, the use of dry, saturated steam is preferable, as it has a higher latent thermal energy compared to wet steam or superheated steam, and is therefore more efficient to reduce the microbial load and also to reduce the problem of product agglomerations which is known to frequently happen in the case of bulk products in large treatment chambers and in the case of mixed products.

### STATE OF THE ART

The objective of the present invention is therefore intended to search for an optimized solution for an autoclave for the combined pasteurization and blending of organic materials in powder form or as particulates, with specific reference to food products in bulk; a research of prior art was carried out in patent literature, which has put in evidence some relevant documents:

### D1: WO2009003546 (Perren et al.)

### D2: GB1243823 (Loedige et al.)

### D3: GB2249706 (Lucas)

### FR2680637 (Antonini et al.)

### D5: WO9843682 (Forberg)

### D6: EP1508364 (Taniguchi)

### D7: JP2005334854 (Nakano)

### D8: EP1205112 (Dreano et al.)

### D9: GB1445942 (Nestle)

### D10: GB2302258 (Fantozzi et al.)

### D11: EP1450868 (Howe et al.)

D1 describes a pasteurization or sterilization process of food products in particulate form which involves the preheating of the product in a separate vertical vessel and to a temperature close to the treatment temperature, the pasteurization being performed with steam and the removal of the water on the particle surface by means of a vacuum pump; the machine for this treatment is of the vertical vessel type with an internal helicoidally shaped agitator. The vessel has a double jacket with cavity for its heating and cooling, and it is also fitted with multiple accessory systems capable to achieve a vacuum, to increase the internal pressure, and to introduce steam. D2 proposes a steam sterilization process in a closed, horizontal cylindrical chamber, for powder or small particle materials, which also involves a continuous agitation or mixing of the product during the different phases of the treatment; D3, instead, describes a machine for the steam sterilization of animal feed, with a closed, horizontal cylindrically shaped chamber which is equipped with a loading and unloading gate for the product, on opposite sides, being also fitted with a system of internal conveying of a helicoidally shaped screw type on the longitudinal axes, in a way which allows a continuous treatment.

D4 proposes a method of pasteurization and sterilization of agro-food products in bulk by means of a machine with an open chamber allowing a thermal treatment, in a horizontal, elongated vessel, where on the feeding side the material is continuously introduced and moved by a vertically and horizontally vibrating table; D5 describes a method of steam sterilization of materials in granulate form or small particles, which are agitated in a first chamber by two parallel rotors with horizontal shafts, and exposed to steam, and subsequently transferred by gravity to a chamber below where they are moved by an endless screw. D6 proposes a steam agitator-pasteurizer where the agitating device is centrally mounted in a chamber along the longitudinal axis; the said chamber is fitted with a surface heating system by liquid and it is also fitted with a system to filter the material at the exit of the chamber.

D7 proposes a horizontally mounted mixing tank for food products which is fitted with two parallel paddle agitators inside the tank, which is emptied by tilting by means of rotation on said axis, being hinged to the sides; D8, instead, describes a cylindrical vessel with a hermetically closing door for the processing of foods in brine, capable of rotating on its own axis and also when tipped, and being internally fitted with a mixing blade which spans radially over the whole length until the basis.

### State of the Art which is Closest to the Invention

D9 proposes a sterilization process of solid materials of small particles in a rotating vessel which moves the product during a thermal treatment through steam, under aseptic conditions. D10 describes a system for the reduction of the bacterial load in organic materials of the type of herbs or spices, by means of a rotating and conditioning tank, shaped like a double truncated cone symmetrically opposed and having the tips at the opposite ends; opposite in such a way as to allow separately the loading, on the top, and unloading, at the bottom by gravity, being also pivoted to the sides on the horizontal centerline to rotate during the treatment; in particular, the said vessel has a double jacket with cavity for its heating or cooling, being also provided with multiple accessory systems allowing to obtain a vacuum, increasing the internal pressure, and recuperate aromas during processing.

D11 describes a method and its corresponding equipment for the reduction of the bacterial load in organic materials by means of a continuous flow of saturated steam inside a hermetically closed chamber, and where the temperature and the pressure are controlled and correlated according to the tables of saturated steam, also known in physics as Saturated Steam Tables. The treatment process includes the following phases: a) introduction of the material into the chamber, b) selection of the desired temperature according the said material, c)selection of the correlated pressure, d) create the vacuum, e) introduction of steam until the pressure reaches the selected value, f) maintain the pressure and temperature values during the correct treatment time. To speed up the process, in an alternative configuration, there is a preheating phase.

### Disadvantages

Indeed, is has been known that the major part of the described solutions present some disadvantages or at least they have limitations.

In D1 difficulties in the cleaning of the machine are revealed because of the difficult accessibility and also problems to remove the product from the pasteurization vessel, the opening being of a small diameter; furthermore, the machine allows stirring the product in case it remains stuck inside the vessel, which causes mechanical damage to the product itself. D2 does not provide the use of saturated steam nor the preheating of the product, the vessel being fitted with a double jacket only insulated but not temperature controlled; a mixing mechanism is integrated which is difficult to clean and can damage delicate products; furthermore, there is no drying capability integrated which makes it necessary to perform such function in a second external vessel.

D3 does not involve the use of saturated steam, but performs the sterilization of the product with wet steam having the particularity to cook the product and to gelatinize its starch content, which reveals that such a treatment is not equivalent to the present invention because it results in an excessive change of the product and also will damage the product due to the mechanical action of the screw; also in D4 and D5 the product is moved by a screw which can cause damages to delicate products and results in difficulty in cleaning the vessel; it is also obvious that the moving system cannot be used as a blender and that the product needs a drying phase subsequent to the treatment. D6 does not work in saturated steam conditions and it is also obvious that the function of the agitator improves the fluidity of the product to be treated, but may result in the breakage of delicate products and is difficult to clean. Finally, it is clear that D7 and D8 provide a correct blending of the product but not a pasteurization.

D9 is a cooking system which does not involve saturated steam and is not suitable for low moisture food products; furthermore it results in mechanical damages of delicate products and is difficult to clean. D10 performs the thermal treatment of the product by means of heating and cooling the double jacket purely through convection; there is no direct contact of the steam with the product and the efficiency of the treatment is limited. D11 is a static process without blending under saturated steam conditions, with a very long treatment time in order to allow the steam to penetrate into the product and with the possibility that this is achieved in a non-homogeneous way due to the high humidity without the possibility of drying; such a system favors the formation of lumps of the material and is obviously less efficient for the industrial treatment of food products with a low moisture content and has no blending capacity where a high level of quality and capacity is required, as it is possible with the present invention.

With reference to the objective of the present invention, the following disadvantages and limitations found in the known solutions are particularly noticeable: the high risk of cross-contamination, the limited efficiency and the long duration of the treatment, the difficult control of the process parameters and, especially in the cases of not using saturated steam and vacuum drying, the change of the product quality following the treatment and the particularly high moisture absorption, the impossibility to get a perfectly blended product at the end of the cycle which is ready to be packed without agglomerates, the difficult integration of the different functions in one compact and automated installation. Generally, it was also found that the blending and agitating systems to fluidize the product and to improve the treatment using paddles or screws in a mechanical movement cause damage to delicate products of the type of bulk food products in powder form or particulates with a low moisture content, such as for example and not limited to, the cereal mixes, flakes or blends of dehydrated vegetables. Furthermore, there are large and complex surfaces to be cleaned with numerous crevices where the dirt is difficult to remove, especially in the presence of mobile elements such as paddles and spirals of the mixers.

From these introductory remarks we can conclude that it is certainly important to identify alternative solutions which are more effective.

### Short Description of the Invention

This and other scopes are achieved with the present innovation according to the characteristics of the included claims solving the explained problems by means of a rotating autoclave (10) using saturated steam in an inclined vessel (110, 151), for the pasteurization and sterilization of particulate products (100) with a low moisture content, combined with a blending function to optimize the treatment and to deliver a product which is ready for packaging. A particular, automated treatment process (20) is used, which has at least the following operational sequential phases: a loading Phase A, a tempering Phase B, a pre-vacuum Phase C, a pasteurization Phase D, a post-vacuum Phase E, a venting Phase F, and an unloading Phase G.

### Scopes

In this way, through the appreciable creative contribution, the effect of which constitutes an immediate technical progress, several advantages are achieved.

A first scope consists in the realization of a machine of the type of an autoclave for organic bulk materials in powder form or particulates, which combines at the same time the functions of blending and pasteurization-sterilization, the said autoclave being of a rotating type and also optimized for the use of saturated steam, according to a particular treatment process; particularly, the said combination of functions is achieved in a way which results in an extremely homogeneous and efficient treatment of the whole load, without agglomerates. Such a rotating autoclave allows at the same time the execution of treatment cycles which conventionally are called pasteurization, or cycles which conventionally are called sterilization.

A second scope consist in the realization of a rotating autoclave using saturated steam which allows to achieve a perfectly blended product at the end of the cycle, which is ready to be packed, also in case of different materials and/or different types and particle sizes, without agglomerates; this advantage is particularly achieved for bulk products of a low moisture content, including in powder form.

A third scope consists in the realization of a rotating autoclave using saturated steam which reduces the risk of cross-contamination and allows a quick and efficient cleaning of the surfaces which are in contact with the organic, treated material, facilitating the removal of dirt; such characteristics, in particular, refer to the potential contamination of organic material which will be introduced in the subsequent treatment cycle, also in relation with the known problem of allergens as it happens frequently when changing products.

A fourth scope, directly related with the preceding scopes, consists in the realization of a particular, integrated system to inject steam and extract a vacuum, that uses the same channel for conveying both alternating flows, being fixed and concentric to the axis of rotation of the vessel and fitted with a self-cleaning filter; in particular, the system is optimized in a way which allows to reduce the risk of contamination and to handle the steam injection and the drawing of vacuum in an integrated way, to avoid impacting the effectiveness of the treatment in spite of the complex and mutually interdependent effects.

A fifth scope consists in the realization of a combined blender and pasteurizer-sterilizer in a way to optimize the treatments and to allow for a significant saving of floor space, of weight, of building and environmental cost, of time, and of the overall processing cost; the two functions, in fact, take place at the same time in the same vessel, integrated in a compact way and sharing various accessory systems and components.

A sixth scope consists in the realization of a rotating autoclave for the pasteurization-sterilization which allows an integrated and optimized control of the complete treatment cycle.

Another scope consists in the realization of a machine of the autoclave type for organic bulk materials with a low moisture content, in powder form or as particulates, which is extremely versatile and customizable regarding its use according to specific treatment cycles, executing at the same time the thermal treatment and the blending of the products.

A further scope intended to achieve a particular manufacturing process adapted to pasteurize-sterilize mixtures of particulate products with a low moisture content that cannot be treated with currently known systems in an effective way, allowing high production volumes and low costs. In particular, differently from all prior art where paddles or screws are used to mix and fluidize the products, this invention has an effective blending capacity without breaking even very fragile product particles. Therefore, it is possible to use this invention for the blending and pasteurization-sterilization of relatively fragile products, like e.g. cereal mixes, flakes, or dehydrated vegetable mixes.

These and other advantages will appear from the following detailed description of some preferred embodiments with the aid of the attached schematic drawings, whose details of execution are not to be considered limitative but only illustrative.

### CONTENT OF THE DRAWINGS

FIG. 1: is an orthogonal view, in a vertical section, of the autoclave object of the present invention, where a dashed line and the letters A or B indicate enlarged details referring to the single enlarged detail views, as shown in FIGS. 2 and 3.

FIG. 2: is an enlarged view of the detail A, as shown in FIG. 1, which refers to the rotary coupling with double channel for the thermal control of the cavity of the double jacket.

FIG. 3: is an enlarged view of the detail B, as shown in FIG. 1, which refers to a fixed coupling, single-channel, supplying the treatment chamber with the steam injection and the extraction of a vacuum, this view showing also the detail C which refers to the enlarged section of the filter.

FIG. 4: is a simplified schematic of the treatment process which is the object of the present invention.

FIG. 5: is a double diagram which, as an example, shows in a graphic way the relation of the temperature values and the pressure during the Phases B-F of the treatment process as shown in FIG. 4, where the parameters are particularly optimized for the pasteurization of a blend of cereals containing oatmeal, raisins, and pieces of walnuts.

FIG. 6: is reporting the results achieved through the pasteurization treatment as shown in FIG. 5, referring to a series of nine repeated test cycles under the same conditions, and where the reduction of the bacterial load in the product is expressed on a logarithmic scale.

### PRACTICAL EXECUTION OF THE OBJECT OF THE INVENTION

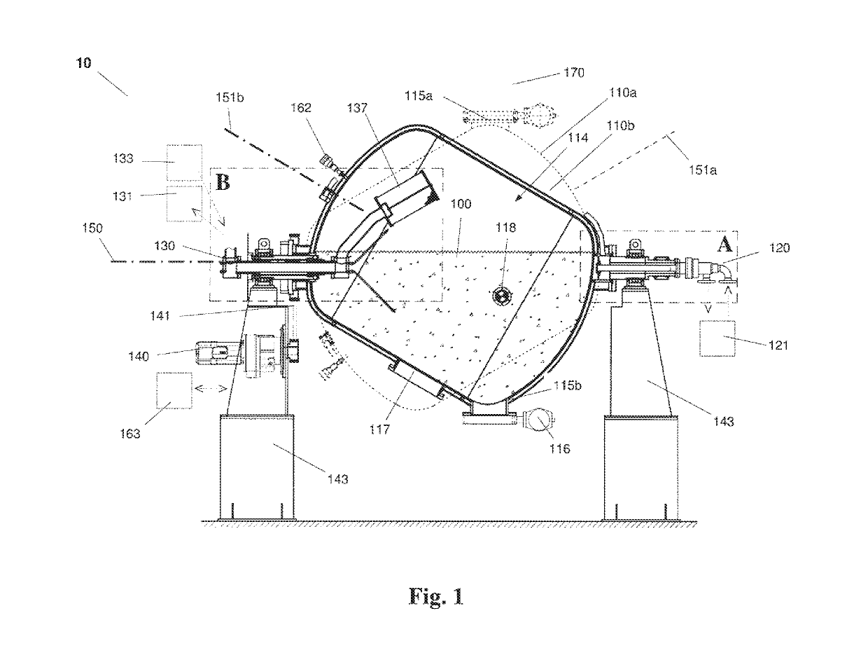

Also with reference to the Illustrations (FIGS. 1-6), the invention describes a machine of the autoclave type using saturated steam, capable of achieving the scopes described above of which, in particular, the optimization of the industrial process of pasteurization-sterilization and the blending, also in combination, homogenizing the treatment and eliminating the formation of agglomerates, reducing significantly the risk of cross-contamination of the treated materials, facilitating the cleaning and having the capability of treating fragile materials without damaging them. This is made possible by means of an innovative apparatus of the type of a rotating autoclave (10) using saturated steam; such equipment is hereinafter called autoclave for descriptive simplicity although advantageously it comprises multiple devices and functions that make it more complex, compared to autoclaves of the conventional type. Additionally, in order to achieve these purposes using said autoclave (10) a particular processing method (20) has to be used, which consists of a sequence of operative phases (Phases A-G) and sub-phases, as described below.

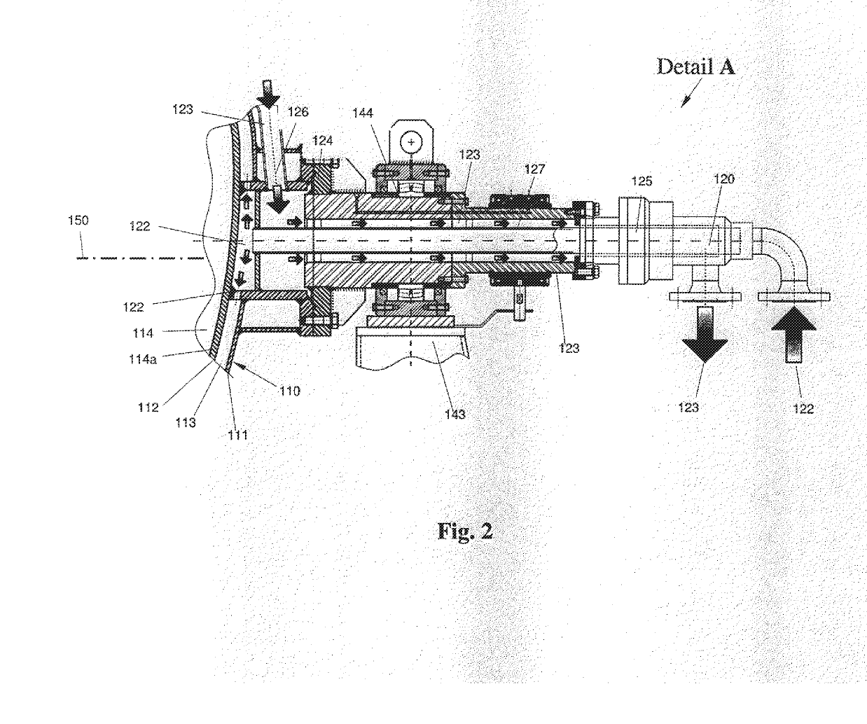

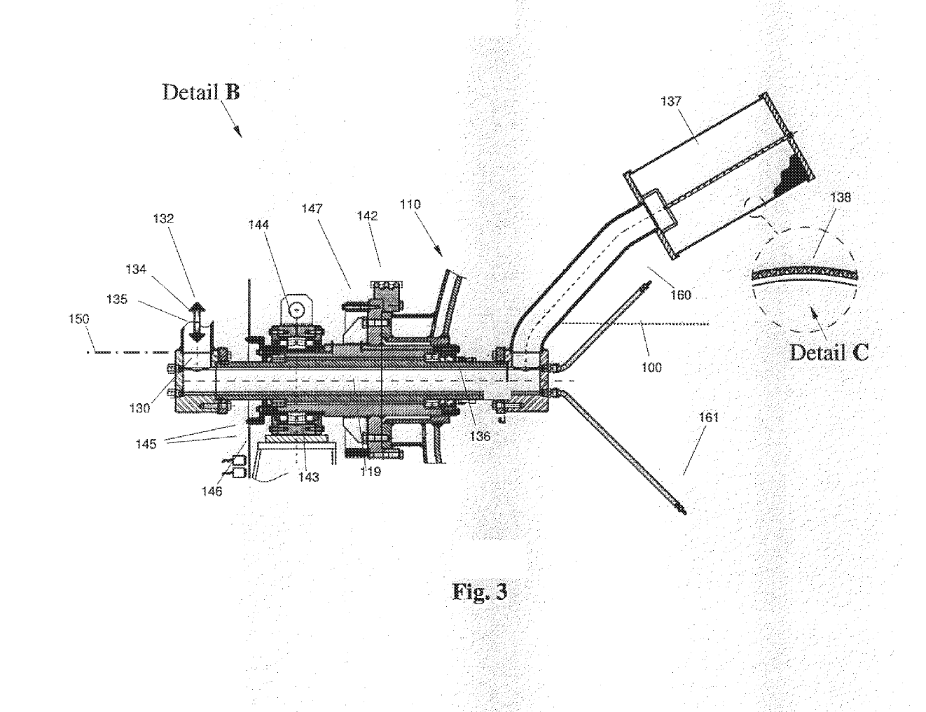

The said rotating autoclave (10) has a treatment chamber (114) of the product (100) inside a cylindrical vessel (110) which can be hermetically closed, controlled and rotated, of a type which allows to create inside various conditions of pressure, and temperature with respect to the external ambient conditions. In the preferred embodiment (FIGS. 1-3), in particular, the said vessel (110) has a double jacket (111, 112) with a cavity (113) that is temperature controlled with a fluid, for example hot water or steam, and is fixed on an inclined longitudinal shaft (151a-b) in such a way as to permit a balanced rotation around the horizontal axis (150), said vessel (110) being supported laterally at the couplings (120, 130) through which the fluids controlling the cavity of the double jacket (113) (FIG. 2) and the chamber (114) (FIG. 3) are conducted in a centralized way through the said rotation axis (150) to allow the free and independent rotation of the vessel (110) during the treatment cycle. Such rotation of the vessel (110, 150), in particular, is taking place in parallel to the temperature control of the cavity of the double jacket (113) and also to the extraction of the vacuum or the injection of steam into the chamber (114).

The internal surface of the said vessel (110) is smooth and free from protuberances in order to facilitate the cleaning, a mechanical polishing being preferable on all the internal surfaces, like for example the surface (114a) which looks to the inside of the chamber (114) in a way to avoid that the treated product (100) sticks to these surfaces and to facilitate the total unloading of the vessel.

In order to facilitate the blending and the fluidization during the treatment, the said vessel (110) has its longitudinal axis (151) inclined at an angle between 30° and 40° with respect to the horizontal axis (150), in such a way as to combine the gravitational mass of the product (100) with a balanced rotation of a slow type, for example between 2 and 15 rpm. This inclined configuration (151) also facilitates the operations of loading and unloading through the opening (115) with a valve (116) which is arranged at the highest point (110a, 115a, 151a) in the loading position, or at the lowest point (110b, 115b, 151b) in the unloading position, resulting from a rotation of 180° on the horizontal axis (150) to change from one position to another position automatically. The said vessel (110) is preferably made of stainless steel of a thickness commensurate with the size in function of the conditions of internal pressure, with an internal capacity of the chamber (114) between 200 and 30,000 liters with pressures between −1.0 barg and +1.5 barg. Additionally, there is a sight glass (118) and also a manhole (117) to access the interior of the autoclave for maintenance and cleaning.

The rotating autoclave (10) object of the present invention, in addition to the said vessel (110) in the preferred embodiment, has at least the following elements:
lateral supporting legs (143) consisting, at the top, of conventional supports to allow rotation (144) of the vessel (110) on the horizontal axis (150);
a system for the rotation of the vessel (110) to the horizontal axis (150) including a motor (140) of the type of electric motor with inverter allowing to adjust the rotation speed between 0 rpm and 15 rpm, and also including the devices of transmission of motion such as, for example, chain system (141) and cog-wheel (142);
a system for the temperature control of the cavity (113) (FIGS. 1, 2) of the double jacket that quickly and in a controlled way allows the treatment chamber (114) to operate under saturated steam conditions, i.e. in optimal conditions for the maximum effectiveness of pasteurization, including at least an external generator of heat with circulator (121) with a conditioning fluid (122, 123), for example steam or water, the same being injected through rotating coupling with double channel (120). For example, the black steam produced by a quick reaction steam generator can advantageously be used as a temperature control fluid.
a rotary coupling with concentric (FIG. 2) double channel (120) of the type named Johnson Fluiten, allowing the release into the cavity (113) of the incoming fluid (122) that heats the inner wall (112) in direct contact with the product (100) and evacuates it back into the rotary coupling externally through an outer tube (123, 126);
means to supply (130-4, 137) the said treatment chamber (114) alternatively steam, or drawing a vacuum through a fixed coupling (130), consisting also of: a filtering system (137, 138), a vacuum pump (131-2), a steam generator (133-4),
a joint service device (130) for the chamber (114) (FIGS. 1, 3) disposed symmetrically on the horizontal axis (150) with respect to said rotary coupling (120), being of single-channel and bi-directional type for the injection of steam or sterile air, or for the extraction of vacuum; said coupling (130) being fixed, integrated with the support structure and terminating inside the chamber (114) with a particular filtration system (137, 138) and also a closure element to the mechanical seal (136) that allows the rotation of the tank (110) under positive or negative pressure;
a filtration and diffusion system of a self-cleaning type, inside the chamber (114), including a particular filter of the type called wedge wire screen (137, 138) allowing the effective filtration of the air or the steam leaving the chamber (114) during the vacuum extraction phase and also allowing the passage of steam and sterile air in such a way that an effective cleaning of the filter is performed simultaneously. Such filtration system prevents the movement of the product (100) into the various auxiliary components and also maintains itself clean and ready for the next phase, with maximum efficiency and safety, without interfering with the rotation of the vessel (110);
an external vacuum pump (131, 132), preferably of the type referred to as single stage or liquid ring vacuum pump, suitable to realize the vacuum in the chamber (114) in a short time and with high pressure so as to prepare the product (100) to receive quickly and homogenously the steam;
a steam generator (133, 134) made of stainless steel for the injection of clean steam into the chamber (114) through a tube (135), the said coupling (130) and said filter (137) for the purposes of the treatment of the product (100) in saturated steam conditions. Optionally, it is also possible to use a carbon steel steam generator producing steam of the type used to heat the cavity of the double jacket, in combination with a steam converter in stainless steel, converting the black steam to clean steam preferably made from osmotic water; this clean steam is used as process steam in direct contact with the products;
sensors controlling the rotation of the vessel (110), also controlling the position of the vessel for loading and unloading (145), rotation locator (146) around the horizontal axis (150), and a collector for data transmission (147) for example of the type with electric rotary coupling connected to the pressure transducer (162) and to the end switch of the loading-unloading valve (116);
a pneumatic coupling (127), also for the actuation of the loading and unloading;
are also included, though not shown in the figures of reference: an external filter of the sterile type and equipped with a valve for injection into the chamber of ambient filtered air, a motorized valve of the type of proportional valve to precisely control the steam injection into the autoclave, a heat exchanger connected to a water chiller which supplies the cooling fluid to the vacuum pump for optimal performance, a heat exchanger for cooling and condensing the steam and humid hot air from the autoclave with a system of condensate evacuation and a system of condensate recovery to reduce water consumption during the cycle, an external air compressor of the type called oil-free screw compressor, feeding filtered, compressed air into a tank, preferably equipped with an UV-light to prevent any microbial build-up, conventional systems of safety for pressure vessels;
an electronic controller (163) of PLC type (Programmable Logic Controller), for the automated control of the autoclave (10) and of the whole procedure of processing (20), for example the controller called Siemens Simatic S7-300-CPU314C, including a card for its connection to an Ethernet network and a router for its connection to the internet, for the purpose of remote assistance and trouble shooting, and also being connected to a PC with a touch screen on which the programs for the treatment process are installed and for the remote assistance, such as customizable software called Simatic WinCC RT. The said PLC can be mounted in the electrical control board.

The said PLC (163) is then managed using software of the conventional type that is customized and parameterized as a function of said rotating autoclave (10) and of the processing method (20), which is specific to the product to be treated (100), or to the blend of products, and depending on the particular treatment to be performed. More in detail, the invention provides that the said software is customized and parameterized in such a way that an entire treatment cycle is initiated manually by the operator choosing quickly and intuitively by said touch screen, connected to the PLC, a specific treatment program, which comprises the complete set of instructions and parameters that characterize the particular procedure to be performed. In order to allow the proper management of the industrial production and quality, the said program, when executed, will then be combined with a number of standardized routine information, for example, but not limited to, the date, the lot number, the name of the processed product, the name of the operator, the sequential number of the cycle.

The said PLC (163) allows to control precisely and automatically all stages of the cycle, the said software parameterized according to the critical variables of the process, which are at least the temperature of the product, the temperature of the chamber, the temperature of the double jacket, the pressure, the duration of pasteurization phase and the speed of rotation, all monitored and controlled by the said sensors in a way to quickly adjust the process according to the measured values. At the end of the cycle it is then possible to automatically get all the values recorded during the execution of the process, including eventual deviations from the set values.

It is noted that the said rotary autoclave (10) integrates in a complex manner multiple functions, also with fixed and rotating elements which are combined in the said rotary coupling (120) and the said fixed supply coupling (130) to the treatment chamber both being centralized on the horizontal axis of rotation (150). In particular, thanks to said single filter (137) which is fixed and shaped in a way to adjust to the particular design of the inclined and balanced rotating vessel (110), it allows its free rotation independent from the steam injection (131-2), the vacuum extraction (133-4) and the temperature control of the cavity of the double jacket (120-1), allowing these functions to be executed separately or jointly in accordance with the procedure determined and laid down in each individual treatment program.

The rotating autoclave (10), built as described above, allows preferably to perform optimized pasteurization treatments in a temperature range between 60° C. and 99° C., at a low pasteurization pressure, but also to perform a sterilization at a temperature range between 100° C. and 121° C., at a higher pressure; said treatments, being combined with a perfect blending performed at a rotation speed between 2 and 15 rpm, also provides a blended, pasteurized finished product (100) ready to be packed.

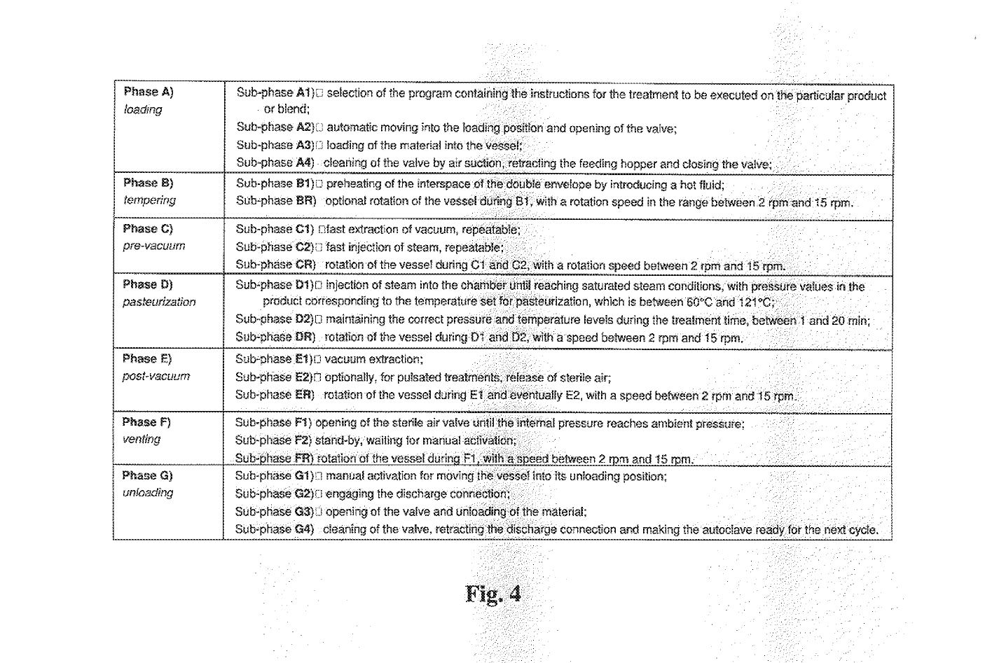

The said rotating autoclave (10), built as described above, allows to reach on an industrial scale the intended claims, by means of a particulate treatment process (20) of the type of sequential operational phases (Phases A-G), including the respective sub-phases (Sub-Phases A1-G4), being the said process (20) described hereafter and also summarized in the simplified schematic diagram of reference (FIG. 4).

The Phase A), otherwise called loading phase of the material or the materials, if of multiple types, includes at least the following sub-phases:
Sub-phase A1)□ Initiate the process by selecting, for example by means of a conventional touch screen connected to said controller of a PLC type (163), a specific program that activates sequentially the said Phases and Sub-phases by properly setting the values for the main parameters that characterize the treatment such as, at least: the product temperature, the chamber temperature, the temperature of the double jacket, the pressure, the duration of the pasteurization phase, the rotation speed; the said program, therefore, substantially corresponds to the collection of standardized information and settings which, depending on the particular product and the treatment to perform, automatically and accurately adjusts the autoclave (10). Additionally, through the said PLC, there are at least standardized recordings such as, for example, cycle number, lot number, description of the treated product, and all the treatment parameters which are critical for the effective pasteurization of the product, like temperature, pressure and time values.
Sub-phase A2)□ The rotating autoclave (10) is automatically rotated with the vessel (110a) in the position for loading the material (100), the main opening (115a) positioned on the top (151a) (FIG. 1) directly underneath the external feeding hopper (170) which preferably is of a type of discharge by gravity, or by pressure, and automatically opening the valve (116) integrated in the main opening (115a);
Sub-phase A3) □ the feeding hopper (170) is engaged and then the material to be treated, also called batch, is discharged. The batch may consist of one or several different products which, generally but not exclusively, are loaded sequentially;
Sub-phase A4)□ once the loading is finished, the cleaning of the opening and the valve (115, 116) is performed by means of suction of air, so as to eliminate the residual material, withdrawing subsequently the feeding hopper (170) and closing the valve.

The Phase B), otherwise called tempering is designed to preheat the material (100) to a temperature level and for a duration according to the program, also with blending, if required by the type of product (100), which can be constituted by a single material or an aggregate of materials, in order to standardize and accelerate the processing, said Phase B comprising at least the following sub-phases:
Sub-phase B1)□ automatic preheating, according to the program referred to in A1, of the cavity (113) of the double jacket (111, 112) by means of a hot fluid such as steam or water, the product (100) being heated through convection by the inner wall (112);
Sub-phase BR)□ automatic rotation of the vessel (110) around the horizontal axis (150), according to the program referred to in A1, resulting in the product (100) being blended in a particularly efficient manner, with the purpose of homogeneously heating the product thanks to the particular inclined configuration (151a-b) and to the limited rotation speed which could be set between 2 rpm and 15 rpm. In the preferred embodiment, the said sub-phases B1 and B2 are performed simultaneously and during the complete time set in the program referred to in A1.

The Phase C) otherwise called pre-vacuum, allows to raise in a short time the temperature of the product (100) in the chamber (114) by means of a rapid vacuum extraction (131-2) which is followed by a sudden injection of steam (133-4). Both the said vacuum extraction and the said steam injection pass exclusively through the single filter (130, 137), which is of the type called wedge wire screen being bidirectional and self-cleaning (FIGS. 1, 3). In this phase, the vessel (110) rotates at a set speed set by the program in such a way as to favor the homogeneous elimination of the air and optimizing the penetration of the steam through the entire volume of material. Said Phase C includes, therefore, at least the following sub-phases:
Sub-phase C1)□ fast and automatic evacuation of the air in the chamber (114), though resulting in a vacuum according to the target pressure value set in the program referred to in A1;
Sub-phase C2)□ automatic injection of steam inside the chamber (114), until the target pressure value set in the program referred to in

A1 is reached;
Sub-phase CR) automatic rotation of the vessel (110) according to the program referred to in A1, during C1 and C2;
   In particular, the said sub-phases C1 and C2 can be repeated several times, if required by said program. In this regard, it is noted that the Phase C always begins with the vacuum extraction, referred to in the Sub-phase C1, and ends with the steam injection, referred to in Sub-phase C2, in order to optimize the steam penetration and to properly condition the product (100) for the subsequent Phase D. Depending on the specific material to be treated and the specific treatment to be performed, therefore, the said step C is performed once or may be repeated with the objective to get the proper penetration of steam into the whole volume of product. The control of this Phase C is reaching target pressure values set by the program for both the vacuum phase and the injection of steam, with the objective to successfully conclude this Phase C in the shortest possible time in such a way that the product is heated with a minimum pick-up of moisture.

The Phase D), generally called pasteurization phase, is the main phase for the purpose of reduction of the bacterial load and provides that, starting from the conditions obtained from the previous Phase C, there is an automatic injection of steam into the chamber (114) until the pressure corresponding to the conditions of saturated steam and, consequently, when the product (100) reaches the temperature of pasteurization as defined by said program referred to in A1, it starts measuring the actual duration of the treatment. The adjustment of the variable parameters, then, during this Phase D, is done according to the known properties of the saturated steam, i.e. by setting the values of pressure in the chamber (114) which correspond to the particular temperature value of the product (100), possibly also adjusting the temperature of the fluid in the cavity (113, 121-3) of the double jacket. Said Phase D includes at least the following sub-phases:
Sub-phase D1)□ automatic injection of the steam into the chamber (114) until reaching, in the product (100), the correct temperature level for its pasteurization or sterilization;
Sub-phase D2)□ automatically maintaining, for the whole duration of the heat treatment, the correct level of pressure in the chamber (114) and the pasteurization temperature in the material (100);
Sub-phase DR) automatic rotation of the vessel (110) according to the values set in the program referred to in A1, during D1 and D2.
   In particular, the invention provides that the correct level of pasteurization temperature, as described above, is between 60° C. and 121° C., while the correct duration of this heat treatment is typically but not limited to between 1 min. and 20 min., these parameters being set and automatically controlled in the program referred to in A1, depending on the specific material or combination of materials treated and the specific treatment envisaged. As an example, but not limited to it, the defined treatment values for a conventional pasteurization treatment of a type of a blend of oatmeal with raisins and walnut pieces are a pasteurization temperature of 95° C. for a duration of at least 8 min.; furthermore, for a conventional sterilization treatment of vanilla powder, the defined level of temperature is 115° C. for a duration of at least 12 min.

It is also noted that, thanks to the integrated control system mainly consisting of the said temperature and pressure sensors (160-2) and of the controller (163) that automatically adjusts the correct levels of pressure as a function of the set temperature, the said autoclave (10) is able to accurately maintain the interior of the chamber (114) in conditions of saturated steam for a long time, also by means of a valve of a modulating type that controls the amount of steam injected in the chamber, and for this purpose, there are continuous adjustments of steam injection through the filter (137) and also of the temperature of the cavity (113) between the double jacket. Additionally, it is noted that the said autoclave allows to create and maintain such conditions in an optimized way even during the blending action, being designed to rotate independently with respect to the various accessory devices which control the conditions inside the chamber (120-3, 130-4, 137). The autoclave rotates at a speed set automatically by the program referred to in the Sub-phase A1, in such a way as to optimize the exposure of the product homogeneously to said saturated steam; at the end of the set time for the pasteurization or sterilization treatment, it automatically switches to the subsequent Phase E.

The Phase E) called phase of post-vacuum, is designed to cool and dry the material (100), its duration is defined by the program referred to in A1 depending on the product to be treated. The said Phase E includes, therefore, at least the following sub-phases:
Sub-phase E1)□ automatic creation of a vacuum inside the chamber (114), according to the pressure target value defined in the program referred to in A1.
Sub-phase E2)□ optionally, in a pulsated drying configuration, ambient air is released through an external sterile filter by opening a valve, until a pre-selected target pressure has been reached.
Sub-phase ER) automatic rotation of the vessel (110) according to the program referred to in A1, while E1 and eventually during E2 with the purpose to homogenize the treatment.
   In particular, for the said Phase E there is the option to choose between a post-vacuum of a pulsating type where vacuum cycles as referred to in the Sub-phase E1 are alternating with the injection of sterile air as referred to in the Sub-phase E2 for a pre-selected duration, and a simple post-vacuum of a fixed type where the defined minimum pressure target has to be reached as referred to in Sub-phase E1 and then maintained during a predefined time. The vacuum is pulled through the wedge wire screen (137) to avoid that powders are carried out of the autoclave (110) by the pump (131). When introducing ambient air into the autoclave, this ambient air will pass through the external sterile filter to avoid any contamination of the product from the air. Due to the pressure differential, negative pressure inside the autoclave and ambient pressure outside, the air will naturally flow into the autoclave. Once the defined post-vacuum time is reached, the system will automatically switch to the subsequent Phase F.

The Phase F), otherwise called venting phase, is suitable to return the chamber (114) to atmospheric pressure in order to perform the unloading of the product. This Phase F, therefore, includes at least the following sub-phases:
Sub-phase F1):□ automatic opening of the valve connected to the external sterile filter, naturally releasing sterile air into the chamber (114) still under vacuum conditions;
Sub-phase F2): reached the level of ambient pressure, for safety reasons the autoclave remains on stand-by waiting for the manual activation of the next and final Phase G;
Sub-phase FR) automatic rotation of the vessel (110) according to the program referred to in A1 during F1.

The Phase G), otherwise referred to as discharge phase, for safety reasons, is manually activated by the operator, preferably by means of a touch screen connected with the controller (163), in a way to initiate the following sub-phases:
Sub-phase G1)□ the autoclave (10) automatically rotates in its unloading position (110b) (FIG. 1), in a way that the main opening (115b) is positioned at the bottom, over an external discharging recipient;
Sub-phase G2)□ engages the said connection, which can be according to the requirements of the customer alternatively a direct discharge by gravity into a hopper placed below the main opening (115b) or into a dense phase pneumatic transport line;
Sub-phase G3)□ opens the valve (116) integrated in said opening (115b) and the complete discharge of the treated material (100) is performed;
Sub-phase G4)□ finished discharging, the said valve (116) is cleaned by an air suction system from residual material, and subsequently the autoclave (10) is ready for the next cycle according to the said treatment process (20).

Consequently, the organic bulk material of a low moisture content in powder form or as particulates, which is loaded into the chamber (114) of said rotating autoclave (10) can be industrially processed according to the treatment process (20) described above, solving advantageously the above described problems and achieving the intended purposes. Furthermore it is noted that, thanks to the particular constructive configuration of said rotary autoclave (10), it is possible to perform combined blending and pasteurization-sterilization treatments of any organic bulk material, powders or particulates, with a low moisture level, the process being customizable with wide variability of all operational parameters characterizing the said treatment system, with particular reference to pressure, temperature and uniformity of application, and also according to new programs for new products. Additionally, the invention allows to optimize the cleaning significantly reducing the chances of contamination and for this purpose, for example, is fitted a particular bidirectional self-cleaning filter (137) and also it is expected that when materials are changed an automatic cleaning cycle is performed which can be enhanced with an intensive wash down by means of an opening of the type manhole (117) that allows periodically to enter inside the chamber (114) for a complete and easy sanitation of all surfaces that are in contact with the material, being particularly smooth and without internal moving elements or protruding parts.

The invention described above also enables advantageously to treat the said material (100) in an automated way, achieving a high quality at reduced operating cost, being also suitable for high production volumes.

By way of a non-limiting example, follows an example of a practical application of the present invention (10, 20) which is related in particular to a pasteurization treatment optimized for a mix of cereals including oatmeal, raisins and pieces of walnuts. For this purpose, the said process (20) provides the said Phases and Sub-phases defined as follows:

Phase A) of loading:
Sub-phase A1) Selection of treatment program on the touch screen;
Sub-phase A2) Moving in the loading position and opening of the valve, identical for all products;
Sub-phase A3) Standard loading/starting procedure, identical for all products;
Sub-phase A4) Cleaning and closing of the valve, retracting the feed hopper, identical for all products;
   Phase B) of tempering:
Sub-phase B1) Pre-heating to target product temperature: 30° C.
Sub-phase BR) Rotation speed: 8 rpm, during B1;
   Phase C) of pre-vacuum:
Sub-phase C1) Initial pre-vacuum with target level of −0.910 bar;
Sub-phase C2) Steam injection with pressure target level of 1 bar;
Sub-phase CR) Rotation speed: 8 rpm, during C1 and C2;
   Phase D) of pasteurization:
Sub-phase D1) Stop injection of steam at target pressure of 1 bar to reach 100° C., the specified pasteurization temperature;
Sub-phase D2) Regulation of the temperature of the double jacket and of the chamber pressure to maintain saturated steam conditions during the specified pasteurization time of 9 minutes;
Sub-phase DR) Rotation speed: 8 rpm, during D1 and D2;
   Phase E) of post-vacuum:
Sub-phase E1) Pulling a vacuum by opening the vacuum valve until the set target pressure of −0.820 bar has been reached;
Sub-phase E2) Continue to pull a vacuum during a set target time of 10 minutes;
Sub-phase ER) Rotation speed: 8 rpm, during E1 and E2;
   Phase F) of venting:
Sub-phase F1) Introduction of ambient air through a sterile filter into the autoclave until the pressure inside the autoclave reaches ambient pressure;
Sub-phase F2) Stand-by waiting for the manual activation;
Sub-phase FR) Rotation speed: 8 rpm, during F1;
   Phase G) of unloading:
Sub-phase G1-G4) Standard discharging procedure, identical for all products.

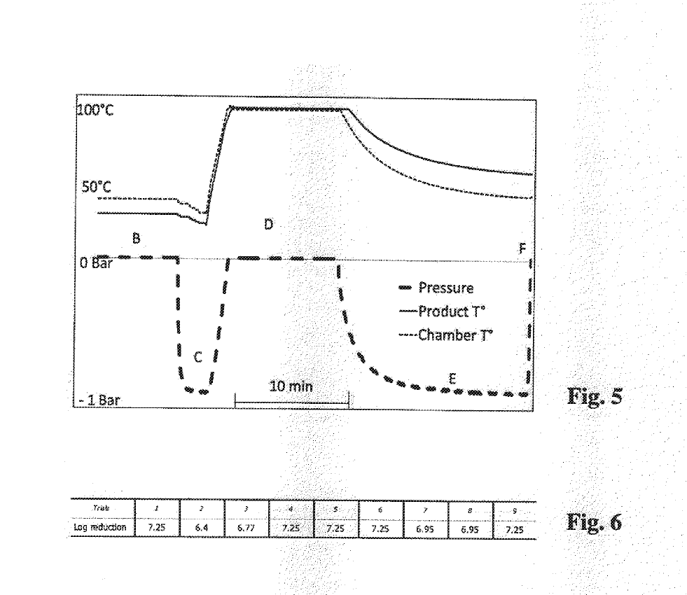

With the rotary autoclave (10) realized as described by the present invention and used according to the said method of processing (20), it is possible to achieve an increased performance in pasteurization and blending compared to conventional solutions. By way of non-limiting example, are shown graphically (FIG. 5) the parameters of the pasteurization cycle described above that is optimized for the pasteurization of a mix of cereals including oatmeal, raisins and pieces of walnuts, being depicted a double diagram that puts in direct relation the values of temperature and pressure during said Phases B-F. In particular, it was found that such a process of pasteurization is extremely effective and also repeatable; in more detail, a series of nine cycles of pasteurization has been performed of said cereal mix, inoculated with Enterococcus faecium and was measured the effectiveness of the said treatment in terms of Log reduction (FIG. 6), on a logarithmic scale that defines the value of the pathogenic microbes eliminated, such results being comprised between a minimum of 6.4 and a maximum of 7.25 and average equal to about 7, which corresponds to a reduction of the bacterial load of about one hundred times compared to the values that are usually achieved with conventional solutions of pasteurization used today, which correspond to approximately 5 Log reduction.

Additionally, it is noted that the parameter that measures the quality of mixing and which is conventionally called blending performance, with respect to said mix of cereals including oatmeal, raisins and pieces of walnuts and particularly treated as described above (FIG. 5), is excellent, and so are the moisture values, measured as a percentage in the samples taken during Phase G unloading and during Phase A loading.

### LEGEND

(10) rotating autoclave for saturated steam conditions understood as a complex and automated equipment which integrates multiple components and functions which are combined between them as described by the invention;
(100) product or a mix of products to be treated, (110) rotating vessel with inclined axis, (110a) vessel in its loading position, (110b) vessel in its unloading position, (111) external wall, (112) inner wall, (113) temperature controlled cavity of the double jacket, (114) inner treatment chamber, (114a) inner surface of the chamber, (115) opening for loading-unloading, (115a) opening in loading position, (115b) opening in unloading position, (116) valve, (117) manhole, (118) sight glass, (119) support legs of the vessel;
(120) double channel rotary coupling, (121) external heater with fluid circulation system, (122) inlet for hot fluid, (123) return flow of fluid, (124) connecting flange with the vessel, (125) fixed support, (126) fluid return pipe, (127) pneumatic coupling, (130) single-channel fixed coupling, (131) external vacuum pump, (132) flow of vacuum extraction, (133) external steam generator, (134) flow of steam injection, (135) connection pipe, (136) mechanical rotating sealing element, (137) fixed filter of a type wedge wire screen, (138) opening;
(140) gear box, (141) transmission chain, (142) cog-wheel, (143) support structure, (144) fixed support for rotation, (145) fixed sensors for positioning, (146) rotating element of reference, (147) collector for data transmission;
(150) horizontal axis of rotation, (151) longitudinal axes of vessel, (151a) longitudinal axis in loading position, (151b) longitudinal axis in unloading position;
(160) chamber temperature probe, (161) product temperature probe, (162) pressure transmitter, (163) electronic controller of PLC type;
(170) external feeding hopper;
(20) treatment process object of the invention.

1. An automatic rotating autoclave (10) for use in saturated steam conditions for the pasteurization-sterilization of low moisture products (100) of particulate type, with the added function of agitator-blender, being the complete treatment chamber (114) a cylindrical vessel (110) with hermetic closure of a type of double jacket (111, 112) with cavity (113) and moved by an electrical motor; wherein
said vessel (110) has the longitudinal axis (151) inclined to the axis of horizontal rotation (150), where the said treatment chamber (114) of said vessel (110) has the inner surface (114a) smooth, without protuberances or elements for the mechanical agitation of the product (100), and where the said vessel (110) has at least:
means for supporting and rotating it (140-4, 150);
means for the temperature control (120-3) of the said cavity (113) of the double jacket through a rotary coupling (120) positioned in connection with the said axis of rotation (150);
means to supply (130-4, 137) the said treatment chamber (114) alternately with steam injection or vacuum extraction through a fixed coupling (130) which is positioned in connection with the said axis of rotation (150), on the opposite side with respect to the said means of temperature control of the said cavity (113, 120-3), also with a system of filtration and diffusion (137, 138) inside the chamber (114) which is connected with the said fixed coupling (130) and of a self-cleaning type;
sensor means, allowing the detection of the rotation and the position of the vessel (110) with respect to the horizontal axis (145, 150);
an electronic controller (163) of a PLC type which is connected in an integrated way at least to said means for the temperature control (113, 120-3), for supplying (114, 130-4), for supporting and rotating (140-4) and at least to said sensor means of temperature and pressure (160-2) and rotation (145) in order to monitor and automatically adjust the values of pressure and temperature in the chamber (114) so as to achieve and maintain the correct treatment conditions of the product (100) even during the rotation of the vessel (110) and with saturated steam, the said controller (163) adapted to manage automatically the operation of the autoclave (10) and the whole treatment process (20).

2. The rotating autoclave (10) according to claim 1, wherein said vessel (110), is rotatable on the horizontal axis (150) of rotation in such a way that it is taking its loading position with the opening (115) in the highest point (110a, 115a, 151a) or its unloading position with the opening (115) in its lowest point (110b, 115b, 151b), being the said opening (115) also fitted with a valve (116); the said vessel (110) which acts as an agitator-blender combining a slow speed of rotation with the gravitational mass of the product (100); and where said means (120-3) for the temperature control of the cavity (113) comprise an external heater with circulator (121) of hot fluid (122, 123) that introduces through said rotary coupling (120), which is of the dual channel concentric type, and where said means (130-4) to service the treatment chamber (114) comprise at least one vacuum pump (131-2), a steam generator (133-4), the said fixed coupling (130) which is of a single-channel and bi-directional type and also provides for a closure element to the mechanical seal (136) in such a way as to allow the simultaneous and independent rotation of the pressure vessel (110) during the vacuum extraction and during the steam injection, and where the said system of filtration and diffusion is achieved with a filter (137, 138) of the wedge wire screen type, and where the said sensors (160-2) combine the sensors of pressure and temperature of the fluid (122-3) in the cavity (113) of the double jacket, and where said controller (163) electronically integrates the said devices (116, 121-3, 131-4, 140-2160-2) in such a way as to automatically manage the operation according to a particular program that is selected when the cycle is initiated in function of the product (100) to be treated and the treatment to perform; the said program, allowing to instruct the said controller (163) with the correct operating parameters in such a way that the correct saturated steam conditions are achieved and maintained in the whole chamber (114) during the rotation.

3. The rotating autoclave (10) according to claim 1, wherein the said vessel (110) is made of a food grade stainless metal having an inner capacity of the chamber (114) in the range between 200 and 30,000 liters; and where said vessel (110) has the said longitudinal axis (151) with an inclination in the range between 30° and 40° with respect to the horizontal axis (150) of the autoclave (10); and where the said speed of slow rotation is in the range between 2 rpm and 15 rpm.

4. Treatment process (20) with sequential operative phase for the pasteurization-sterilization of powders or particulate low moisture food products (100) by means of a rotating autoclave as referred to in claim 1, said process (20) providing in a logic sequence at least (Phases A-G):
Phase A) loading phase, including at least: Sub-phase A1), initiation of the predefined program from the PLC and standardized recording; Sub-phase A2), going into loading position with opening of the valve; Sub-phase A3), loading of the material in the vessel; Sub-phase A4), cleaning of the
valve by air suction, retracting the feed hopper and closing the valve.
Phase B) tempering phase, allowing the preheating of the material, including at least: Sub-phase B1), heating of the cavity by introducing a hot fluid, steam or water; Sub-phase BR), possible slow rotation of the vessel (110) during B1.
Phase C) pre-vacuum phase, allowing to increase in a short time the temperature of the chamber by means of a rapid vacuum extraction followed by a rapid steam injection, until the predefined pressure level is reached, and where the vacuum and the steam pass through the same single bi-directional and self-cleaning filter, and where the slow rotation favors the homogeneous evacuation of the air in the product and optimizes the contact with the steam, being such Phase C executed once or also in a repeated way according to the program, always beginning with the vacuum and ending with the steam injection, said Phase C including at least: Subphase C1), realization of a rapid vacuum; Sub-phase C2) rapid injection of steam; Sub-phase CR), slow rotation of the vessel (110) during C1 and C2.
Phase D) pasteurization phase, where to maintain the correct pasteurization or sterilization temperature under saturated steam conditions are provided adjustments by the controlled injection of steam into the chamber (114) and the temperature control of the cavity (113), said Phase D including at least: Sub-phase D1), steam injection into the chamber (114), where pressure values on the material (100) are achieved which correspond to the pre-selected temperature, which is in the range between 60° C. and 121° C.; Sub-phase D2), maintaining the pressure and temperature levels during the whole defined duration of the treatment, which is in the range typically but not limited to between 1 min. and 20 min.; Sub-phase DR), slow rotation of the vessel (110) during D1 and D2.
Phase E) post-vacuum phase, allowing to cool and dry the material, including at least: Sub-phase E1), extraction of vacuum; Sub-phase E2), eventually, in the treatments of the pulsated type, it is also provided the release of air through a valve with a sterile air filter; Sub-phase ER), slow rotation of the vessel (110) during E1 and eventually E2.
Phase F) venting phase, allowing the chamber to return to atmospheric pressure for discharging, including at least: Sub-phase F1), opening of the valve releasing sterile air into the chamber until reaching the ambient pressure; Sub-phase F2), stand-by and waiting for manual activation; Sub-phase FR), slow rotation of the vessel (110) during F1.
Phase G) unloading phase, manually activated, including at least: Sub-phase G1), moving of the vessel (110) into the unloading position; Sub-phase G2), engagement of the discharge connection; Sub-phase G3), opening of the valve and unloading the material; Sub-phase G4), cleaning of the valve, and making the autoclave (10) ready for the subsequent processing cycle.

5. The rotating autoclave (10) according to claim 2, wherein the said vessel (110) is made of a food grade stainless metal having an inner capacity of the chamber (114) in the range between 200 and 30,000 liters; and where said vessel (110) has the said longitudinal axis (151) with an inclination in the range between 30° and 40° with respect to the horizontal axis (150) of the autoclave (10); and where the said speed of slow rotation is in the range between 2 rpm and 15 rpm.
